(19) United States
(12) Patent Application Publication
(10) Pub. No.: US 2020/0371744 A1
(43) Pub. Date: Nov. 26, 2020

(54) METHODS AND SYSTEMS FOR RECORDING AND PROCESSING AN IMAGE OF A TISSUE BASED ON VOICE COMMANDS

(71) Applicant: KangHsuan Co. Ltd, Taipei City (TW)

(72) Inventor: Wei-Hsuan LIAO, New Taipei City (TW)

(73) Assignee: KangHsuan Co. Ltd, Taipei City (TW)

(21) Appl. No.: 16/881,040

(22) Filed: May 22, 2020

(30) Foreign Application Priority Data
May 23, 2019 (TW) 108117892

(51) Int. Cl. G06F 3/16; G10L 15/26; G16H 10/60; G16H 30/20; G16H 30/40; H04M 11/10

(52) U.S. Cl. CPC G06F 3/167; G10L 15/265; H04M 11/10; G16H 30/20; G16H 30/40; G16H 10/60

(57) ABSTRACT

Provided herein are methods and systems for recording and processing images of a tissue by use of voice commands. The method includes steps of: (a) recording a video of the tissue; (b) capturing a target image from the recorded video; and (c) storing the captured target image and a voice information corresponding thereto as a medical record in a database. The present method is characterized in that at least the steps (b), (c) or both is/are executed under a voice command. Also provided herein is a system for implementing the present method.

### CROSS-REFERENCE TO RELATED APPLICATION

This application relates to and claims the benefit of TW Patent Application No. 108117892, filed May 23, 2019, the content of which is incorporated herein by reference in its entirety.

### BACKGROUND OF THE INVENTION

### 1. Field of the Invention

The present disclosure relates to the information processing system and method, and in particular to the system and method for recording information related to the image based on a voice command.

2. Description of Related Art

Medical record, particularly, records that are images of a lesion, is essential to the diagnosis of a disease. Not only does it keep the record of the disease, but also allows medical practitioner to prescribe suitable treatments to the lesion.

In clinical practice, medical records oftentimes are not recorded simultaneously during the surgery or treatment. For instance, while operating an endoscope, a physician oftentimes is unable to take down medical records as both of his/her hands are occupied with the instruments. Thus, he/she would revert to record his/her findings during the surgery and/or treatment afterwards (i.e., after the diagnosis and/or operation) based on the photograph(s) or video taken during the operation, and his/her memory of the instance. Such practice inadvertently renders the medical records related to the diagnosis and/or treatment prone to incompleteness, or worst, errors.

Another important issue generally associated with making diagnosis and/or treatment with an endoscope is that the operator needs to decide on the spot the location of the endoscope in the body, and/or the type of the lesion through the observed images. If the medical practitioner mistakenly determined the location, it would lead to misdiagnosis, or applying inappropriate or unnecessary therapy.

In view of the foregoing, there exists in this art a need of an improved method and/or system for a medical practitioner to take medical records while operating a medical instrument, particularly, an endoscope.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

One aspect of the present disclosure aims to provide a method for recording and processing images of a tissue, comprising the steps of:
(a) recording a video of the tissue;
(b) capturing a target image from the recorded video of the step (a); and
(c) storing the target image captured in the step (b) and a voice information corresponding thereto as a medical record in a database;
   wherein, the steps (b) and (c) are respectively executed via voice commands.

According to one specific embodiment of the present disclosure, the voice command comprises an action command; and a text command comprising the voice information configure to be converted into a text.

According to one optional embodiment, the action command is configured to dictate an image-recording device to execute the step (b); dictate a controller to store, delete, select, and/or record the target image; perform the voice-to-text conversion to convert the voice information comprised in the text command into the text; or associate the target image with the text.

According to optional embodiments, the text command comprises at least one classification information selected from the group consisting of disease, shape, size, color, time, treatment, surgery, equipment, medicine, description and a combination thereof. In one embodiment, method further comprising identifying at least one historical medical record corresponding to the medical record from the database.

According to another embodiment, the method further comprising the steps of:

storing a plurality of templates in the database, wherein each of the plurality of templates has a first image feature and information corresponding to the anatomical location of the first image feature;

analyzing the target image to determine if it has an image feature at least 90% identical to the first image feature thereby deducing the anatomical location of the target image to be same as that of the first image feature.

In another embodiment of present disclosure, the method further comprising the steps of:

repeating step (b) to capture a plurality of the target images;

analyzing the timing and/or order of the image feature of each target images; and

comparing the first image feature of each template and the timeline that the plurality of the target images appeared in the video to obtain the anatomical location of the plurality of the target images.

In one specific embodiment, each of the templates is a historical medical record and/or a tissue image. Moreover, the image feature may be any one of the shape, the texture, or the color of a cavity of the tissue, or a combination thereof.

According to one specific embodiment, the method further comprises the step of displaying the medical record and the historical medical record according to the anatomical location of the target image in the tissue. In one preferred embodiment, the method further comprises the step of generating a schematic drawing to indicate the anatomical location of the lesion in the tissue.

Another aspect of the present invention is directed to a method for recording and processing images of a tissue. The method comprises the steps of:

(a) recording a video of the tissue;

(b) issuing a first voice command, which comprises a first action command and a first text command;

(c) capturing a plurality of target images from the recorded video of the step (a);

(d) assigning the plurality of target images capture in the step (c) in a group and tagging the group with a text converted from a voice information stated in the first text command;

(e) storing the tagged group of target images in a database; and

(f) issuing a second voice command to terminate the method.

According to one specific embodiment, the method further comprises the steps of:

(g) issuing a third voice command to timestamp the target images to obtain at least one timestamp target image; and

(h) storing the timestamp target image in the database.

Further, in one embodiment of present disclosure, the method comprises the steps of:

repeating the step (g) to produce a plurality of the timestamp target images; and

calculating the interval between any two timestamps.

Additionally, the methods disclosed in accordance with embodiments described above would combine or modify according to actual needs.

On the other hand, another aspect of the present invention is directed to a system for recording and processing images of a tissue. For example, the system comprises an image-recording device, and a controller communicated with the image recording device.

The details of one or more embodiments of this disclosure are set forth in the accompanying description below. Other features and advantages of the invention will be apparent from the detail descriptions, and from claims.

Many of the attendant features and advantages of the present disclosure will becomes better understood with reference to the following detailed description considered in connection with the accompanying drawings.

### BRIEF DESCRIPTION OF THE DRAWINGS

The accompanying drawings, which are incorporated in and constitute a part of the specification, illustrate various example systems, methods and other exemplified embodiments of various aspects of the invention. The present description will be better understood from the following detailed description read in light of the accompanying drawings, where,

FIG. 1 is a block diagram illustrating a system in accordance with one embodiment of the present disclosure;

FIG. 2 is a flow chart illustrating steps of a method for recording and processing images of a tissue under voice commands in accordance with one embodiment of the present disclosure;

FIG. 3 is a schematic drawing depicting a screenshot 300 of a medical record in accordance with one embodiment of the present invention;

FIG. 4 is a schematic drawing depicting a screenshot 400 of a medical record in accordance with another embodiment of the present invention;

FIG. 5 is a schematic drawing depicting a screenshot 500 of retrieving historical target images based on the selected image feature of a target image in accordance with another embodiment of the present invention;

FIG. 6A is a schematic drawing depicting a screenshot 600 of tagged target images in accordance with one embodiment of the present invention;

FIG. 6B is a schematic drawing depicting the table 602 generated in the embodiment of FIG. 6A;

FIG. 7A is schematic drawing depicting a screenshot 700 of structuralized tagged target images 742 another embodiment of present invention;

FIG. 7B is a schematic drawing depicting the table 702 generated in the embodiment of FIG. 7A;

FIG. 8 is a schematic drawing depicting the change in pattern of a status bar 800 along a timeline 810 in response to voice commands 804 and 806 in accordance with one embodiment of the present disclosure;

FIG. 9A is a schematic drawing depicting events occurred in response to a timestamp voice command in accordance with one embodiment of the present disclosure;

FIG. 9B is a schematic drawing depicting a screenshot 900 of timestamp medical records of a colonoscopy examination in accordance with one embodiment of the present disclosure;

FIG. 9C is a schematic drawing depicting the table 902 generated in the embodiment of FIG. 9B;

FIGS. 10A and 10B are screenshots 1000a and 1000b depicting the operation of the present system and/or method in accordance with one embodiment of the present disclosure; and

FIG. 11 is a screenshot 1100 depicting the operation of the present system and/or method in a colonoscopy examination in accordance with one embodiment of the present disclosure.

In accordance with common practice, the various described features/elements are not drawn to scale but instead are drawn to best illustrate specific features/elements relevant to the present invention. Also, like reference numerals and designations in the various drawings are used to indicate like elements/parts.

### DESCRIPTION

The detailed description provided below in connection with the appended drawings is intended as a description of the present examples and is not intended to represent the only forms in which the present example may be constructed or utilized. The description sets forth the functions of the examples and the sequence of steps for constructing and operating the examples. However, the same or equivalent functions and sequences may be accomplished by different examples.

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Unless otherwise required by context, it will be understood that singular terms shall include plural forms of the same and plural terms shall include the singular. Also, as used herein and in the claims, the terms “at least one” and “one or more” have the same meaning and include one, two, three, or more. Furthermore, the phrases “at least one of A, B, and C”, “at least one of A, B, or C” and “at least one of A, B and/or C,” as use throughout this specification and the appended claims, are intended to cover A alone, B alone, C alone, A and B together, B and C together, A and C together, as well as A, B, and C together.

The term “video” as used herein refers to the collection of a plurality of real-time images continuously captured in a period-of-time by an imaging recording device operated by a medical practitioner or physician during a medical examination or a surgical procedure. For example, in an endoscopic procedure, the “video” refers to the video recording during the gastrointestinal endoscopy examination.

The term “target image” as used herein refers to an entire frame in a video, or a part of a frame in a video. In some embodiments, the target image is one frame of a video. In other embodiments, the target image is a small part of a frame of a video, particularly the part selected by the user of the present method and/or system. In specific embodiment, the target image can be any type of graphs obtained from clinical. For example, the target image may be captured from radiography, electroencephalography, electrocardiogram, electromyogram, diagram of sound wave, diagram of flow or endoscopy.

The term “medical record” as used herein refers to a medical record generated by the method or system of the present invention. For example, the “medical record” is directed to a clinical record of a subject generated by the present method or system during a surgery or a medical examination, in which the clinical record includes a target image (i.e., tissue image) and information related thereto, such as the diagnosis, observation, and treatment information orally given by a medical practitioner (e.g., nurses, technician, or physician).

The term “finding” as used herein refers information or fact that has been discovered by medical practitioners or physicians. In one embodiment of present invention, the finding is directed to a pathological condition.

The term “pathological history data” refers to at least one medical record of a subject existing prior to the medical record generated by the present method and/or system.

The term “subject” or “patient” refers to an animal including the human species treatable by the methods and/or systems of the present invention. The term “subject” or “patient” intended to refer to both the male and female gender unless one gender is specifically indicated.

1. General Description of the Present Method and System

To address the need of medical practitioners or physicians to include real-time description and annotations of the observation during a medical examination or surgery that requires taking images of a lesion of a patient, the inventors of the present invention develop a method and a system for recording and processing images of a tissue using voice commands.

Accordingly, the present invention is particularly suitable for surgical operations and/or examinations whose execution required both hands of a medical practitioner. For example, during a surgery, both hands of a physician are often occupied with surgical instruments rendering it difficult for the physician to record in real-time the status of the patient, particularly, the lesion condition observed by naked eyes or with the aid of an instrument (e.g., endoscope). The present invention addresses such need by providing an improved method and/or system allowing a medical practitioner to perform tasks using voice commands. Examples of tasks include, but are not limited to, capturing medical images of a lesion from a video, associating such medical images with the physician's observation of the lesion stated in voice commands, storing the images associated with relevant voice information contained in the voice command into medical records, and/or storing medical records in a storage means.

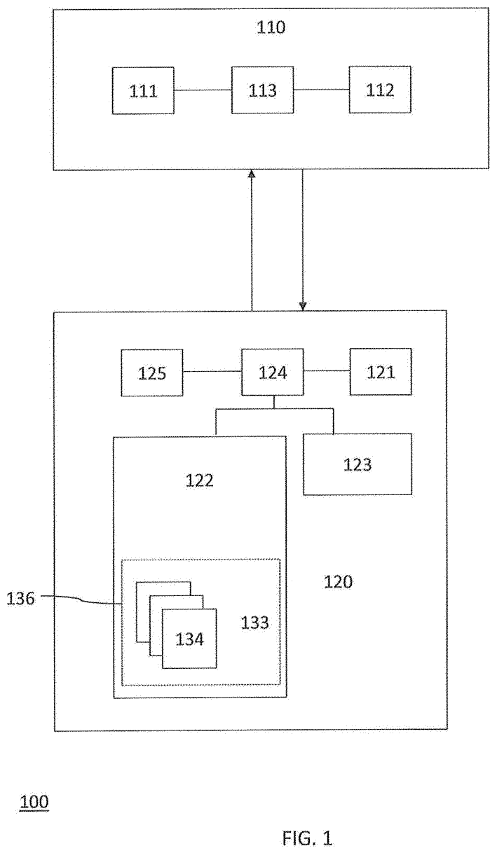

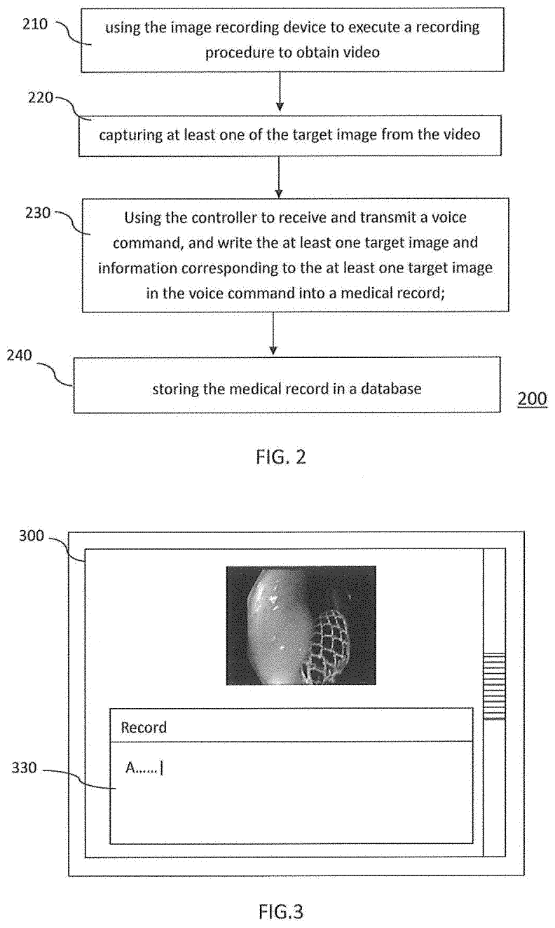

References are first made to both FIGS. 1 and 2, in which FIG. 1 is a schematic diagram depicting a system 100 configured to implement a method 200 of the present invention depicted as a flow chart in FIG. 2.

The present system 100 includes at least an image-recording device 110 and a controller 120 respectively coupled to each other. During a surgery or a medical examination, in which both hands of the attending medical personnel (e.g., a physician) are occupied (e.g., by surgical instruments), the present system may be activated through voice commands. In response to voice commands, the present system 100 may produce a video of a lesion of a subject (step 210), capture desired images from the video (step 220), subsequently process the captured images into medical records (steps 230 and 240), and optionally compared the medical records with historical medical records of the subject.

As depicted in FIG. 1, the image-recording device 110 includes in its structure, a camera 111, a first communication means 112 and a first processor 113 communicatively coupled to the camera 111 and the first communication means 112.

In general, any camera that meets the required specifications of surgery may be used in the present invention. Preferably, the camera 111 is a Charged Coupled Device (CCD) for video recording or image capturing. In one embodiment, the camera 111 is imbedded in an endoscope. The first communication means 112 is configure to transmit and receive data and/or information to and from the first processor 113, which is under the command of the controller 120. According to embodiments of present invention, the first communication means 112 is a communication chip designed to receive and transmit voice commands. Examples of the communication chip include, but are not limited to, Global System for Mobile communication (GSM), Personal Handy-phone System (PHS), Code Division Multiple Access (CDMA), Wideband Code Division Multiple Access (WCDMA), Long Term Evolution (LTE), Worldwide interoperability for Microwave Access (WiMAX), Wireless Fidelity (Wi-Fi) or Bluetooth components. Both the camera 111 and the first communication means 112 are communicatively coupled to, and under the command of, the first processor 113 to perform tasks commanded by the user (e.g., via voice commands). Examples of the first processor 113 suitable for use in the present invention include, but are not limited to, a central processing unit (Central Processing Unit, CPU), programmable general-purpose or special-purpose microprocessor (Microprocessor), digital signal processor (DSP), programmable controller, application specific integrated circuit (ASIC), other similar components, or a combination of any of the above described components.

The image-recording device 110 may be activated manually or automatically (e.g., in response to voice commands of the user) to take images of the lesion and stream them into a video during a surgery or a medical examination. Examples of the image-recording device 110 suitable for use in the present method and/or system include, but are not limited to commercially available optical imaging device, ultrasound imaging device, cardiac catheterization equipment, radiographic imaging device, thermal imaging device electrophysiology device, etc.

The images taken by the camera 111 of the image-recording device 110 are streamed into a video and displayed on a displaying means 125 (e.g., a screen) in real-time basis or afterwards, allowing the user to give oral description to the displayed image, such as the pathological condition of the lesion including the size, color, appearance, inflammation status, etc. Reference is made to the flow chart in FIG. 2 again, after the video is produced, the user may choose a desirable image from the recorded video by issuing a voice command to capture a target image from the video (step 220); then provides relevant description to the chosen target image also through a voice command, and finally commands the chosen target image and the relevant description to be stored together as a medical record in a database (steps 230 and 240).

The controller 120 of the system 100 is designed to receive and process voice commands of the user, such as the steps 220, 230 and 240 of the present method. As depicted in FIG. 1, the controller 120 includes in its structure, a second communication means 121, a storage means 122, an input device 123, a second processor 124, and a displaying device 125. Note that the second communication means 121, the storage means 122, the input device 123, and the displaying means 125 are all under control of the second processor 124. In general, the user uses the input device 123 to input voice commands into the controller 120. Examples of the input device 123 include, but are not limited to, a microphone, a keyboard, a mouse, a touch screen, a pedal, a human machine interface or other communication interface that allows the user to input data through external electronic devices, such as inputting information via Bluetooth from a mobile device like a smart phone, a tablet computer, etc. The hardware of the second processor 124 and the second communication means 121 are similar to the first processor 113, and the first communication means 121, thus description thereto is omitted for the sake of brevity. According to preferred embodiments, the user uses a microphone to input voice commands into the controller 120. The inputted commands are processed by the second processor 124, which then issues instructions to deploy the second communication means 121, the storage means 122, and/or the displaying means 125 into actions, depending on the content of the voice command. The voice command in general includes at least, an action command; and a text command, which is configure to be converted into a text through the action of the action command.

Take the task of extracting a target image from the video as an example, conventionally, a triggering device (e.g., by use of a pedal, a button, a mouse, etc.) may be used to extract or capture a desired image. In the present method, the system 100 extracts a target image from the video in response to a voice command. The voice command is processed by the second processor 124, which in terms will instruct relevant components of the system 100 to act accordingly to complete the task instructed in the voice command. In some embodiments, the target image is an entire frame of the video. In other embodiments, the target image is merely a certain area of a frame (i.e., a part of the frame), in which case, the input device 123 can be used to circle or select an area-of-interest from a frame or an image. As to the task of providing description to a captured image and subsequently store the captured image and the description into a medical record, voice command in this regard is also processed by the second processor 124, which will perform a voice-to-text conversion to convert the descriptive information stated in the voice command into a text, and then store the target image alone with the text as a medical record 134 in the storage means 122. Descriptive information may be tagged on each target image, so that the target image can be classified and retrieved based on the tagged descriptive information. The medical records 134 (particularly those having the same class) stored in the storage means 122 will constitute a database 136 suitable for acting as a resource for machine learning. In a non-limiting embodiment, the present system 100 may be operated by machine learning, in which the large number of medical records 134 stored in the system may serve as the training materials for machine deep learning.

Alternatively or additionally, prior to implementing the method 200 of the present invention, the user may retrieve the patient's prior record (i.e., pathological history) from other resource and input them through the input device 123 upon starting the present system 100. Note that the patient's prior record or pathological history data 133 includes at least one medical record 134 of the patient. In the case when the patient's pathological history data 133 has already existed in the storage means 122 of the present system 100, the controller 120 will retrieve the pathological history data 133 from the storage means 122, then proceed to add new medical record 134 to it after implementing the present method 200.

Furthermore, for identify or analysis the target images, the database 136 has the templates which could be the materials for reference. For example, the templates could be historical medical records and/or tissue images, and those templates may retrieve from other sources (e.g. science database) or already exist in the database 136.

Detail description related to voice commands and capturing a target image of the present method and/or system is provided below.

2. Voice Commands

The voice command of present invention includes at least, an action command and a text command. Examples of the action command include, but are not limited to, commands to instruct the image-recording device 110 to execute a recording or a retrieving action, commands to instruct the controller 120 to store, delete, select, record, associate, or convert information provided in voice into text.

For example, in the case when the user needs to record the features of the tissue displayed on a target image, he/she may issue voice commands to record any one of “the type,” “the shape,” “the morphology,” “the size,” “the classification” of the target; or to record “the result” thereby triggers the present system to execute action(s) stated in the voice command. According to embodiments of the present disclosure, the user may issue more than one voice command. Non-limiting examples of the action command include, but are not limited to, “record/shoot,” “open file,” “terminate record,” “delete record,” “select picture,” “grouping” and “recording the time,” etc. Non-limiting examples of the text command include, but are not limited to, the name or the type of a disease; morphology; size; color; time; treatment; type of surgery; equipment or medicine that has been used; a descriptive information provided by the user; and a combination thereof.

Additionally, or alternatively, the storage means 122 of the present invention may further include a sound wave recognition program and a noise reduction program embedded therein. When the user issues a voice command, which triggers the controller 120 to act accordingly, then, the sound wave recognition program and/or the noise reduction program may be automatically activated; or alternatively, manually activated by the user. The sound wave recognition program serves the purpose of recognizing and identifying the user's voice, and the noise reduction program serves the purpose of rendering the voice of the present user more distinguishable from the background noise or the voice of other user (i.e., non-current user's voice), thereby enhancing the accuracy on the recognition of the inputted voice.

After receiving the voice command, the controller 120 will proceed to determine if the user failed to issue a voice command when a pre-determined period of time has lapsed. If so, the controller 120 will automatically turn off the voice-activated function of the present system, and inform the user accordingly. Additionally, if the sound intensity detected by the controller 120 failed to reach a certain threshold within a pre-determined period of time, the controller 120 will also automatically turn off the voice-activating function of the present system. Alternatively, if the controller 120 received a voice command instructing the controller 120 to “turn off ” the system, it will also proceed to stop all operation accordingly.

Additionally, or alternatively, the voice command may be modified based on the environment or the need of the user. Reference us now made to FIG. 3, which is a schematic drawing depicting a screenshot 300 of a target image and a column 330 for entering text converted from a voice command in accordance with one embodiment of the present invention. The screenshot 300 shows a frame of a video and a column 330 where text will be entered, which is the text converted from a voice command. The user may also switch or scroll screen through voice command(s), or by other means, such as by pushing a button, clicking a mouse, etc. Note that after the controller 120 executes the function to convert a text command into text, the text will automatically shows up in the column 330, thereby allowing the user to verify if the text has included all stated information, or any typos or errors may have resulted from the voice-to-text conversion. Once all stated information has been successfully converted into text and entered into the column 330, the controller 120 may then proceed to inquire the user (either via text appear on the screen or via voice) if the displayed image shall be saved as a medical record. If the entry in the column 330 is incomplete, the controller 120 will also proceed to inform the user accordingly.

3. Target Images and Uses thereof

As defined above, “a target image” captured by the present system and/or method refers to an entire frame in a video or a part of a frame in a video. Accordingly, the target image may be the shape of a cavity of a tissue; or the texture, color, gloss, shape, appearance or morphology of a tissue, and those features could be the image features of the present invention. In the present disclosure, the target image may assist the present method and/or system to determine where (i.e., the anatomical position of a tissue) the target image was captured. To this purpose, the present system and/or method is designed to determine the anatomical position of a tissue or the location of the target image by referencing to the location of the camera 111. Accordingly, the location of the camera 111 may be determined based on the target image per se and the timeline when the image-recording device 110 recorded the video. Alternatively, or additionally, the location of the camera 111 is determined based on the target image(s) and the timeline that the target image(s) appeared in the video. Specifically, location of the camera 111 is determined based on analyzing the timing and/or order of the image feature of each target images appeared in the video.

In another embodiment, the location where the target image captured may be determined based on the target image(s) per se and/or the timeline that the target image(s) appeared in the video, compared with the templates which respectively owns an image feature (i.e. first image feature) corresponding to the tissue and information of anatomical location. Accordingly, the templates are the historical medical records or the tissue images retrieved from the science database or textbook. In optional embodiment, the templates may store in the database 136 or retrieve from other resources, such as external database.

In one specific embodiment of present invention, to achieve the purpose described above, the target image(s) captured by the method/system may be analyzed and extracted the image feature at first; then, the image feature of the target image(s) may be compared with that of the template(s) to obtain the anatomical location result.

According to one specific embodiment, in the step of comparison or analysis the target image to the templates, if the image feature of the target image is at least 80% identical to the first image feature of template thereby deducing the anatomical location of the target image to be the same as that of the first image feature. In one optional embodiment, the percentage of identity between the image features of the template(s) and the target image is at least 80 to 100%, such as 80, 82, 84, 86, 88, 90, 92, 94, 96, 98, 100; more preferably the percentage of identity is at least 90%.

Moreover, the templates may be a series of the images of tissue. For example, for gastrointestinal tract, there are plural images of tissue corresponding to the gastrointestinal tract, leading to a sequential manner of those images. Therefore, the location of the camera 111 is determined based on the target image(s) and the timeline that the target image(s) appeared in the video.

Take enteroscopy examination as an example, the intestine comprises various sections respectively having their own unique structures, shapes, and surface texture, as summarized in Table 1 below.

TABLE 1

| | Name of the section | | | | | | |
|---|---|---|---|---|---|---|---|
| | | sigmoid | descending | transverse | ascending | | |
| | rectum | colon | colon | colon | colon | cecum | ileum |
| Cross- | circle | triangle | triangle | circle | triangle | circle | circle |
| sectional | | | | | | | |
| shape of the | | | | | | | |
| cavity | | | | | | | |
| Shape of the | straight | curved | straight | curved | — | — | — |
| section | | | | | | | |
| Texture or | — | — | — | — | — | — | villi |
| gloss of its | | | | | | | |
| surface | | | | | | | |

Take the sigmoid colon and descending colon as examples, they respectively are triangular in cross-section; thus, the present system and/or method may deduce that the camera 111 is at the sigmoid colon or the descending colon, based on the cross-sectional shape of the cavity, and/or the texture, color of the surface of the tissue appeared on the target image.

Alternatively, or in addition, the location of the camera 111 or the target image may be determined by the user, based on his/her experience, and enters such location into the present system via voice command, the location will appear as an entry in the column (e.g., column 330) on the displaying means 125 (e.g., a screen).

Alternatively, or in addition, the target image may be used to in the comparison of medical records. As described above, the medical record generated from the present method and/or system is stored in the database 135, with new medical records continuously being generated and stored in the database 135, prior medical records become “historical medical records,” in relative to the newest medical record or the one currently in use.

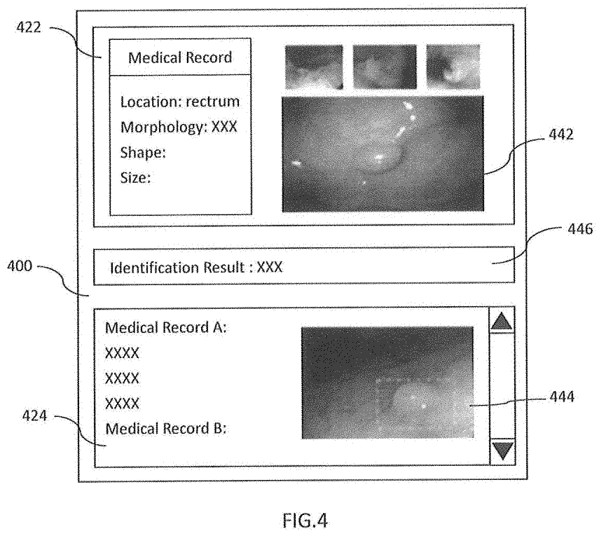

Reference is made to FIG. 4, which is a schematic drawing depicting a screenshot 400 of a medical record 422 and a historical medical record 424. Specifically, with each medical record 422 being made and stored, it became a historical medical record 424 of the subject. Accordingly, the target image 442 becomes the target image 444 in the historical medical record 424. All historical medical record(s) of the subject in the database 136 may be retrieved by the present method and/or system. Additionally, upon capturing the target image 442, the present method and/or system will automatically compare the target image 442 with all historical target image 444 corresponding thereto. Furthermore, the present method and/or system will also determine if the image feature in the historical target image 444 is similar or identical to that of the target image 442, and produce a result 446 that is also automatically displayed on the displaying means 125. The result 446 may also be stored into the medical record 442 via voice command.

Alternatively, or in addition, the present method may further determine if the lesion in the target image 442 is the same or different from that on the target image 444 in the historical medical record 424. To this purpose, all historical medical records 424 respectively containing the target images 444 are retrieved and displayed in accordance with their respective similarities to the lesion in the target image 442. Referring again to FIG. 4, in which the historical target image 444 and the target image 442 are displayed simultaneously on the screenshot 400. In the case when there is not any historical target image 444 may be retrieved and paired with the target image 442, then the lesion on the target image 442 is a new one. Accordingly, the user may issue a voice command to add descriptive information related to the new lesion and store the newly added descriptive information along with the target image 442 as a medical record 422. After the medical record 422 has been saved and stored in the database, the present method may be terminated, also through a voice command, such as “terminate recording”.

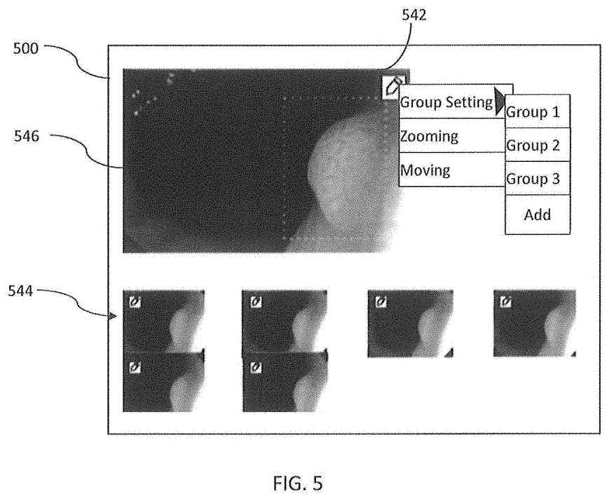

Alternatively, or in addition, instead of comparing the entire frame of an image with that of historical record(s), a part of an image frame designated by the user may be used to this purpose. Reference is made to FIG. 5, which is a schematic drawing depicting a screenshot 500 of a target image 542 selected form a frame 546, and corresponding historic target images 544 in accordance with another embodiment of the present invention. In this embodiment, the user circles or selects an target image 542 (shown in dotted line) from a frame 546 for further comparison. After the user has made the selection, the present system will automatically search the historical medical records based on the target image 546, and proceed to display all retrieved medical records independently containing a historic target image 544 based on their respective similarities with the target image 546. Note that in FIG. 5, the historic target images 544 are displayed from left to right with a decrease in similarity in the target image 542. The step of circling or selecting an target image on an frame may be implemented by voice command or other manners. In addition, it should be noted that in the present method and/or system, the user may retrieve the target image 542 from any historical medical record in the database 136, and then proceed to select a certain area for further analysis as desired.

4. Tagging Target Images

The present invention also characterizes in providing structured medical records, so that they may be displayed in an organized manner. To this purpose, target images are respectively tagged by a descriptive information such as type and/or anatomical location of a pathological finding (i.e., lesion); morphology, pathological status, or clinical signs of the lesion; type of treatment; type of surgery; type of examination; examination result; and etc.

Target images may be tagged by embedding the descriptive information described above directly in the target image or by including the descriptive information as an addition to the target image. In the case when the target image is in PEG format, the descriptive information is directly embedded into the target image. In the case when the target image is in RAW format, then a mapping table is created for the entry of the descriptive information as an addition to the target image. Note that the present method and/or system may choose a suitable way to tag a target image (i.e., to include the descriptive information to the target image) based on the format of the target image. According to preferred embodiments of the present disclosure, the target image is tagged via use of a voice command.

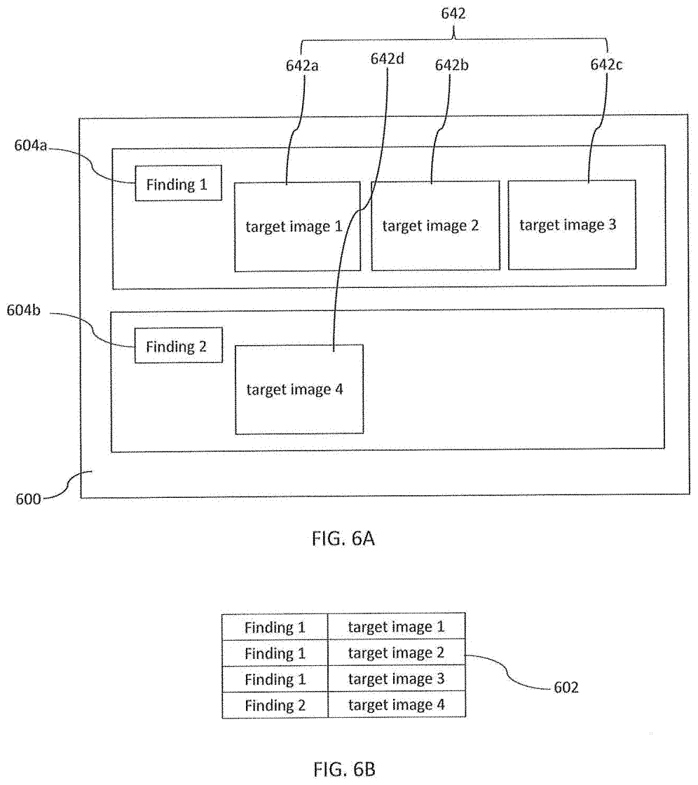

Reference is made to FIG. 6A, which is a schematic drawing depicting a screenshot 600 of tagged target images displayed on a displaying means 125 in accordance with one embodiment of present invention. In this embodiment, the present system provides a list of descriptive information or tags for the user to choose from. The list may include phrases such as, “lesion 1”, “lesion 2”, “undiscovered”, “to be observed”, etc. In the depicted embodiment in FIG. 6A, which is a schematic drawing depicting a screenshot 600 of tagged target images 642 displayed on a displaying means 125 in accordance with one embodiment of present invention. In specific, four target images 642a, 642b, 642c, and 642d were captured from the video; in which target images 642a, 642b, and 642c associated or tagged with the descriptive information of “lesion 1” (604a), and the target image 642d is associated or tagged with “lesion 2” (604b) through voice commands. Further, a table 602 (see FIG. 6B) is generated for accommodating entries of target images and their respective tagged descriptive information (i.e., “lesion 1”, or “lesion 2”). Note that the table 602 is for the use of the present system and/or method, and is not displayed on the displaying means 125.

Additionally, or alternatively, the descriptive information or the tag 604a, 604b may be present in text format. Accordingly, the present method and/or system may display the tagged target images 642a, 642b, 642c, 642d based on their respective tags 604a, 604b, which are in text format. For example, target images bearing the same tag or descriptive information may be displayed under the same tagged text, such as under the text of “lesion 1”.

In non-limiting embodiments of the present invention, each target image may be tagged with one or more tags, including but is not limiting to, “lesion,” “location,” and etc., which may all be integrated into the table 602.

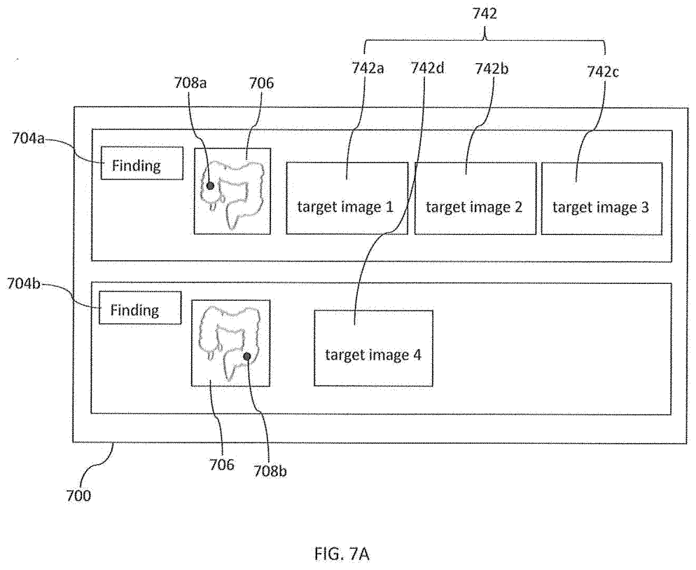

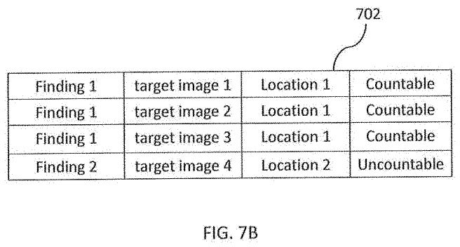

Reference is now made to FIG. 7A, which is a schematic drawing depicting a screenshot 700 of tagged target images 742 displayed on a displaying means 125 in accordance with another embodiment of present invention. In this embodiment, the list of tag provided may further include phases like, “location 1,” “location 2,” “countable,” “uncountable,” etc., in addition to those provided in the table 702 described in FIG. 7B. The “location” refers to the place or area where the lesion appeared in the tissue (e.g., anatomical position) or where the tag image is captured by the camera. The location can be automatically identified by the present system 100 in accordance with the procedures described above in the section of “3. Target images and uses thereof,” thus are not repeated here for the sake of brevity. Alternatively, or in addition, the location may be directly inputted by the user based on his/her clinical experience through voice commands.

Target images 742a, 742b, 742c, and 742d may be classified in accordance with their respective tags. In one example, the target images are classified by the number. For example, when the target images 742a, 742b, and 742c of lesion 1 are solid tumors, which are countable, then these target images 742a, 742b, and 742c of lesion 1 may be further tagged with the phase of “two solid tumors.” A table 702, similar to the table 602 described in FIG. 6B, is also generated to accommodate entries of target images and their respective tagged descriptive information (i.e., “lesion 1”, “lesion 2” “location 1”, “location 2”, “countable”, “uncountable”, and the like) which will also be written into the medical record (see FIG. 7B). Like table 602, the table 702 is also for the use of the present system and/or method, and is not displayed on the displaying means 125.

According to embodiments of the present disclosure, the system 100 will automatically generate descriptive information that corresponds to the target image of lesion 1 (704a) based on the quantity information inputted by the user. For example, when the user input “5” through input device 123, the controller 120 will automatically generate the phase of “5 tumors” on the target image. Additionally, or alternatively, if the number or quantity of lesion 1 entered by the user is greater than 1, the controller 120 will automatically guide the user to choose a suitable sub-description for each lesion. For example, in the case when there are five tumors respectively differ from each other by their appearances, then the user may further classify each tumor by suitable sub-description, for example, lesion 1 may be tagged as “countable” (i.e., in the case of a solid tumor), lesion 2 (704b) may be tagged as “uncountable” (i.e., in the case of an ulcer), etc.

By the tagging process described above, medical records of this invention are structuralized, allowing target images to be classified or organized, and subsequently displayed in accordance with specific tagged text based on the need of the user.

Additionally, or alternatively, the present method and/or system may further generate a schematic drawing to indicate the location of the lesion in the tissue based on the captured tagged target images. Further, a schematic drawing 706 is automatically generated by the controller 120, wherein the location 708a of lesion 1 in the tissue (i.e., anatomical position), which is determined from the places where target images 742a, 742b, and 742c are captured, is marked on the schematic drawing 706 for easy reference of the user (See FIG. 7A). By similar manner, the location 708b of lesion 2 (i.e., anatomical position 708b), which is determined from the place where the target image 742d is captured, is marked on the schematic drawing 706 as well. Therefore, the present method and/or system provides a novel digital medical report, which includes the schematic drawing 706 depicting the anatomical position of a lesion in a tissue, rendering the medical report easier to present to the patient by the medical practitioner.

4.1 Tagging Target Images in Groups

Additionally, or alternatively, to tag target images in a more efficient manner, the present method and/or system further includes a function allowing the user to tag and store a plurality of target images in group(s). To this purpose, a status bar 800 is display on the screen to alert the user that the system and/or method is/are in the state of permitting a plurality of target images to be grouped, tagged, and store in response to voice commands.

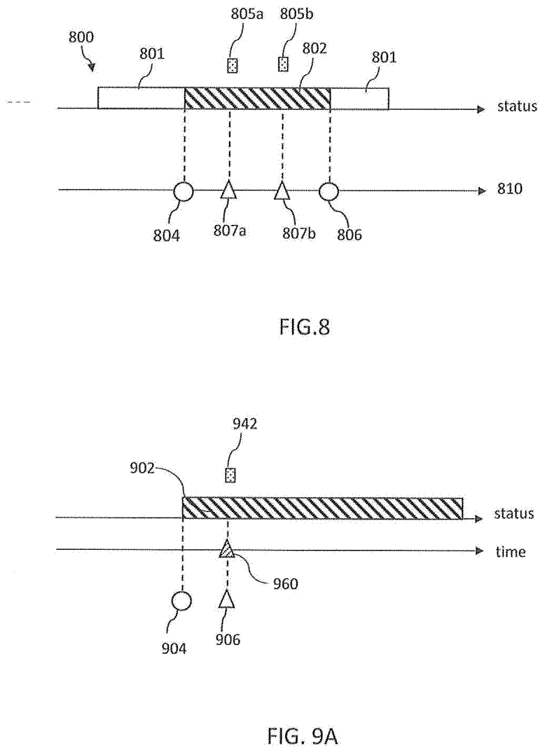

Reference is made to FIG. 8, which is a schematic drawing depicting the change of pattern of a status bar 800 along the timeline 810 in response to voice commands 804 and 806 in accordance with one embodiment of the present disclosure. Upon observing a pathological finding (or lesion) in the produced video, the present method and/or system may automatically bring up a status bar 800 having a first pattern 801 on the screen. Along the timeline 810, upon receiving a voice command 804, the controller 120 of the present system and/or method will instruct the status bar 800 to change pattern from the first pattern 801 to a second pattern 802, alerting the user that each and every target images captured afterwards (i.e., after the issuance of the voice command 804) are automatically grouped together and tag with a descriptive information (e.g., lesion 1) stated in the voice command 804, and then store in the database. A second voice command 806 may be issued later to terminate the first voice command 804. Upon receiving the second voice command 806, the status bar 800 will resume to the first pattern 801. Additionally, or alternatively, the grouping, tagging ad storing target images described herein may be terminated automatically if the controller 120 failed to receive the second voice command 806 within a pre-determined period of time. Note that in the embodiment depicted in FIG. 8, two target images 805a and 805b are captured after the first voice command 804, and are grouped and tagged with the descriptive information stated in the first voice command 804, then store in the database. The target images 805a and 805b may be captured via use of voice command or via any conventional means 807a and 807b (e.g., foot-activated paddle, click of a mouse, etc.). The operation described herein (i.e., grouping, tagging, and storing target images) may be repeated in accordance with the actual need, so that target images are grouped., tagged, and store in the database. By this manner, target images may be tagged in groups, thereby enhances the efficiency of tagging, as well as data entry in the corresponding table (e.g., tables 602 or 702).

5. Timestamp Target Images Via Voice Commands

Additionally, or alternatively, the present system and/or method also includes a function that allows the user to timestamp target images using voice commands. In this embodiment, upon activating the “timestamp” function via a voice command, the present system and/or method will proceed to capture target image(s), timestamp the captured target images and store the timestamp target images as a medical record in the database.

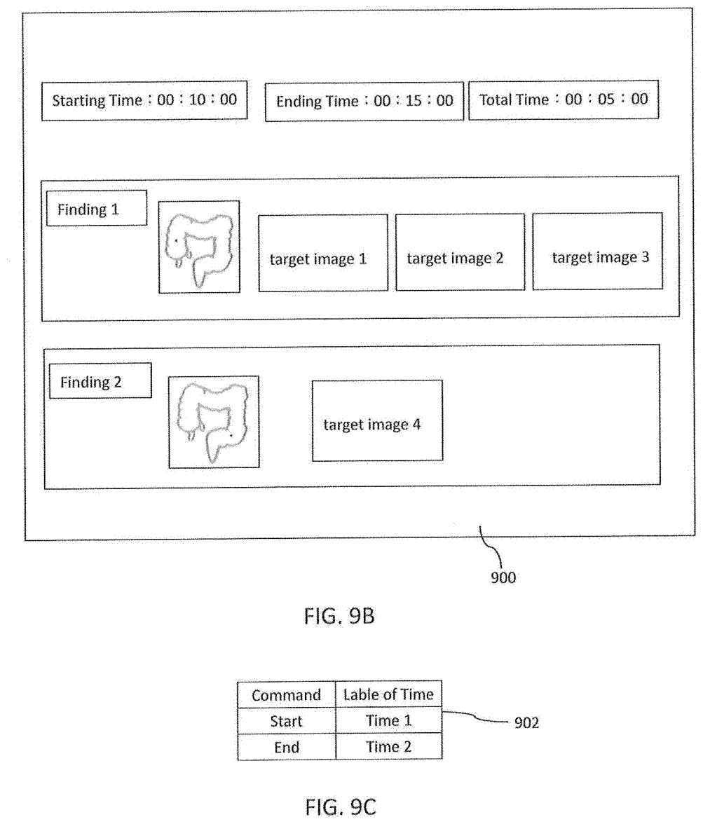

Reference is made to FIG. 9A, which is a schematic drawing depicting events occurred in response to a timestamp voice command. In the depicted example, the user issues a voice command 904—“start timestamp”, which triggers the present system and/or method to start the function of timestamp and into the ready state 902. Then, perform the steps shown as followings: timestamp the target image 942 captured at the time the voice command 906 is issued with the timestamp 960, and store the timestamp target image as a medical record 942 in the database. The voice command 904—“start timestamp” may be repeated in accordance with the actual need of the user. In some embodiments, each timestamp corresponds to one medical record, accordingly, an estimation of the total time required for performing a certain surgery may be calculated by summing up the time between each and every medical record generated during the surgery based on respective timestamp corresponding thereto. In optional embodiments, a medical record may comprise a plurality of timestamps.

The present timestamp function is further described by use of a colonoscopy examination as an example. During such examination, the user (i.e., the physician who operates the enteroscope) first issue a voice command—“start timestamp”, which will automatically trigger the controller 120 to start a timer, and act accordingly (e.g., executing steps as described in FIG. 9A); the user then proceed to place the enteroscope into the patient, and starts giving voice commands, which include but are not limited to, “start timing (or start recording)”, “entering rectum”, “passing ascending colon”, “reversing out”, and “terminate the procedure”. In response to each afore-described voice command, the time and the target image at that moment are recorded or captured thereby producing a target image having a timestamp corresponding thereto. Reference is now made to FIG. 9B, which is a schematic drawing depicting a screenshot 900 of the timestamp and tagged target images of a colonoscopy examination. Upon receiving the voice command—“start timing”, the time at that moment was recorded and shown on the screen as “starting time: 00:10:00”. Similarly, upon receiving the voice command “terminate the procedure” , the time at that moment was recorded and shown on the screen as “ending time: 00:15:00”. In addition, the present system and/or method will also automatically calculate the interval between the two voice commands—“start timing” and “terminate the procedure”, thereby deriving the total time taken to complete the colonoscopy examination, which is also shown on the screen as “total time: 00:05:00”. A table 902 is automatically generated for the entry of each voice command and its corresponding timestamp (see FIG. 9C), and like tables 602, 702, table 902 is for use of the controller 120, and is not displayed on the displaying means 125.

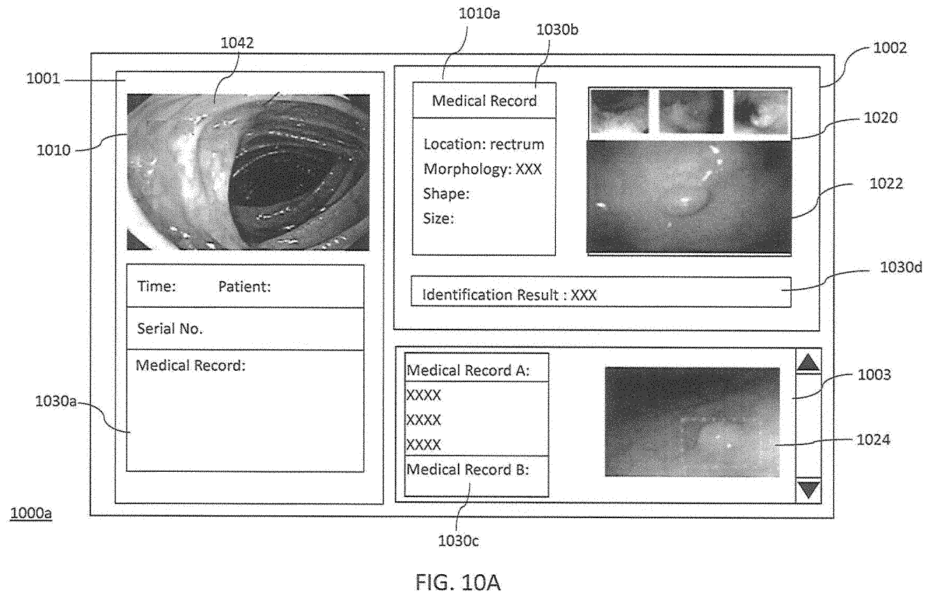

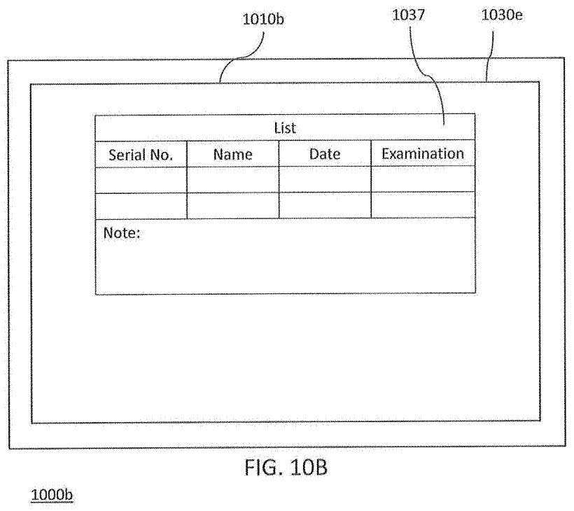

References are now made to FIGS. 10A and 10B, which are screenshots 1000a and 1000b displayed on a displaying means in accordance with one embodiment of the present disclosure. The depicted screenshots 1000a and 1000b may be arranged to be view on the same screen page. Alternatively, they may be arranged to be view on different screen pages, in which case, the user will need to scroll the screen to view both pages; optionally, a call button may be installed on the screen allowing the user to call out the other screen page (i.e., the one not currently in view) for viewing.

As depicted, there are 3 split-screens 1001, 1002 and 1003 on the screenshot 1000a. Specifically, the split-screen 1001 comprises a panel 1010 for displaying a video 1042, and a column 1030a for inputting entries of information relating to the undergoing examination or surgery, including the patient's personal information, medical history and etc. The split-screen 1002 comprises a panel 1020 for displaying one or more target images 1022 captured from the video 1042, a column 1030b for entering text converted from voice commands (e.g., anatomical location of the target images, size or shape of the lesion, etc.), and a column 1030d containing the identification result between the target images 1022 displayed on the split-screen 1002, and the historical target images 1024 in the historical medical record. The split screen 1003 is for displaying one or more historical medical record(s) retrieved from the database, each historical medical record comprise a historical target image 1024, a column 1030c containing text associated with the historical target image 1024. As to the screenshot 1000b depicted in FIG. 10B, it comprises a column 1030e for displaying a list of patients 1037, allowing the use to retrieve patient's information by selecting the patient from the list 1037.

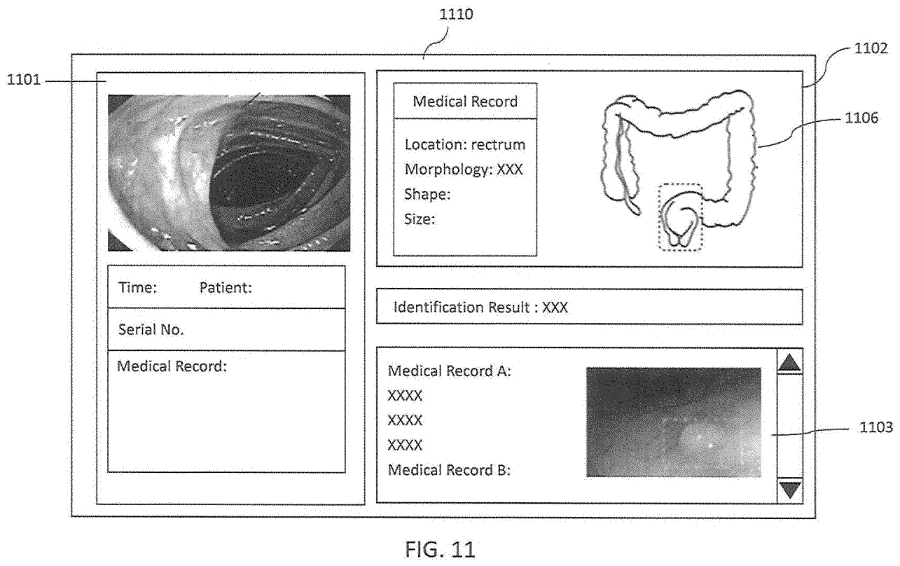

FIG. 11 is a screenshot 1100 depicting the operation of the present system and/or method in a colonoscopy examination in accordance with one embodiment of the present disclosure. Three split-screens 1101, 1102, and 1103 are depicted, in which the split-screen 1101 is for displaying a video and text information related to the examination recorded in the video, the split-screen 1102 is for displaying a medical record comprising a schematic drawing 1106 of the colon, on which the location of the lesion is boxed (shown in dotted line) for easy reference of the user, and the split-screen 1103 is for displaying historical medical records. Note that anatomical location of the lesion is estimated from the location of the camera equipped on the enteroscope in accordance with procedures described above in the section of “3. Target images and uses thereof,” thus are not repeated here for the sake of brevity.

Additionally, or alternatively, all medical records thus produced by the present system and/or method may be viewed directed from the screen or in the form of a print-out. The present system and/or method provide a tool for executing medical examination or surgery through voice commands, thereby allowing medical practitioner to include descriptive information to images of lesion observed during the examination or surgery in real-time basis or afterwards.

It will be understood that the above description of embodiments is given by way of example only and that those with ordinary skill in the art may make various modifications. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method for recording and processing images of a tissue comprising:
(a) recording a video of the tissue;
(b) capturing a target image from the recorded video of the step (a); and
(c) storing the target image captured in the step (b) and a voice information corresponding thereto as a medical record in a database;
wherein,
the steps (b) and (c) are respectively executed via a voice command.

2. The method of claim 1, wherein the voice command comprises an action command; and
a text command comprising the voice information configure to be converted into a text.

3. The method of claim 2, wherein the action command is configured to,
dictate an image-recording device to execute the step (b);
dictate a controller to store, delete, select, and/or record the target image;
perform the voice-to-text conversion to convert the voice information comprised in the text command into the text; or
associate the target image with the text.

4. The method of claim 2, wherein the text command comprises at least one classification information selected from the group consisting of disease, shape, size, color, time, treatment, surgery, equipment, medicine, description and a combination thereof.

5. The method of claim 4, further comprising identifying at least one historical medical record corresponding to the medical record from the database.

6. The method of claim 1, further comprising:
storing a plurality of templates in the database, wherein each of the plurality of templates has a first image feature and information corresponding to the anatomical location of the first image feature; and
analyzing the target image to determine if it has an image feature at least 90% identical to the first image feature thereby deducing the anatomical location of the target image to be same as that of the first image feature.

7. The method of claim 6, wherein each of the templates is a historical medical record and/or tissue image.

8. The method of claim 6, further comprising the steps of:
repeating the step (b) to capture a plurality of the target images;
analyzing the timing and/or order of the image feature of each target images; and
comparing the first image feature of each template and the timeline that the plurality of the target images appeared in the video to obtain the anatomical location of the plurality of the target images.

9. The method of claim 6, further comprising the step of displaying the medical record and the historical medical record according to the anatomical location of the target image in the tissue.

10. The method of claim 6, wherein the image feature is any one of the shape, the texture, or the color of a cavity of the tissue, or a combination thereof.

11. The method of claim 6, further comprising the step of generating a schematic drawing to indicate the anatomical location corresponding to the target image.

12. A system for recording and processing images of a tissue comprising:
an image-recording device configured to execute a recording procedure to produce a video; and
a controller communicatively coupled with the image-recording device and is configured to execute a voice command to,
capture a target image from the video; and
store the captured target image with a voice information in the voice command corresponding to the captured target image as a medical record.

13. A method for recording and processing images of a tissue comprising:
(a) recording a video of the tissue;
(b) issuing a first voice command, which comprises a first action command and a first text command;
(c) capturing a plurality of target images from the recorded video of the step (a);
(d) assigning the plurality of target images capture in the step (c) in a group and tagging the group with a text converted from a voice information stated in the first text command;
(e) storing the tagged group of target images in a database; and
(f) issuing a second voice command to terminate the method.

14. The method of claim 13, further comprising the steps of:
(g) issuing a third voice command to timestamp the target images to obtain at least one timestamp target image; and
(h) storing the timestamp target image in the database.

15. The method of claim 14, further comprising the steps of:
repeating the step (g) to produce a plurality of the timestamp target images; and
calculating the interval between any two timestamps.

16. The method of claim 13, wherein the first action command is configured to,
dictate an image-recording device to execute the step (b);
dictate a controller to store, delete, select, and/or record the target image;
perform the voice-to-text conversion to convert the voice information comprised in the text command into the text; or
associate the target image with the text.

17. The method of claim 13, wherein the first text command comprises at least one classification information selected from the group consisting of disease, shape, size, color, time, treatment, surgery, equipment, medicine, description and a combination thereof.

18. The method of claim her comprising:
storing a plurality of templates in the database, wherein each of the plurality of templates has a first image feature and information corresponding to the anatomical location of the first image feature;
analyzing the target image to determine if it has an image feature at least 90% identical to the first image feature thereby deducing the anatomical location of the target image to be same as that of the first image feature.

19. The method of claim 18, further comprising the steps of:
repeating the step (b) to capture a plurality of the target images;
analyzing the timing and/or order of the image feature of each target images; and
comparing the first image feature of each template and the timeline that the plurality of the target images appeared in the video to obtain the anatomical location of the plurality of the target images.

20. The method of claim 19, further comprising the step of generating a schematic drawing to indicate the anatomical location of the target image.
